(19) United States
(12) Patent Application Publication
(10) Pub. No.: US 2018/0280078 A1
(43) Pub. Date: Oct. 4, 2018

(54) ELECTRICAL SURGICAL SYSTEM FOR CUTTING OR CAUTERIZING TISSUE AND A METHOD OF THE SAME

(71) Applicant: REGENTS OF THE UNIVERSITY OF MINNESOTA, Minneapolis, MN (US)

(72) Inventors: Rei TAMIYA, Tokyo (JP); Bryce C. BEVERLIN, II, Saint Paul, MN (US); Thomas O. VIKER, New Brighton, MN (US); Christie L. TRACZYK, Savage, MN (US); Arthur Guy ERDMAN, New Brighton, MN (US)

(73) Assignee: REGENTS OF THE UNIVERSITY OF MINNESOTA, Minneapolis, MN (US)

(21) Appl. No.: 15/474,406

(22) Filed: Mar. 30, 2017

(51) Int. Cl. A61B 18/14

(52) U.S. Cl. CPC A61B 18/1477; A61B 2017/00738

(57) ABSTRACT

An electrical surgical system 1 for cutting or cauterizing tissue comprises a guide needle 11 having at least one open part 13 and a front end part able to pierce the skin and at least one electrode 12 able to cut or able to cauterize tissue configured to be able to project to the outside of the guide needle 11 and able to be retracted to the inside of the guide needle 11 through the at least one open part 13, in the state where the at least one electrode 12 projects out, the distal end of the at least one electrode 12 being arranged at a position separated from the axis of the guide needle 11.

### FIELD

The present invention relates to an electrical surgical system for cutting or cauterizing tissue and a method of the same.

### BACKGROUND

Carpal tunnel syndrome is treated by carpal tunnel release surgery comprising completely cutting the transverse carpal ligament for lowering the pressure inside the carpal tunnel. To cut the transverse carpal ligament, there exists 1) an open carpal tunnel release surgery where part of the skin of the wrist and palm is cut open in the longitudinal direction to enable the transverse carpal ligament to be viewed from the outside and the ligament is cut and 2) an endoscopic carpal tunnel release surgery where an incision is made in the wrist in the transverse direction and a rod-shaped cutting instrument is inserted from the incision to cut the ligament. In each method, tissue other than the transverse carpal ligament can be damaged over a broad area, so more time is required for post-surgical recovery. Further, there is the possibility of infection from the incision.

Therefore, US 2011/087255 A1 discloses the method of piercing the skin with a hollow introducer having a sharp front end part and inserting the cutting instrument into the body through the introducer so as to reduce the range of damage of the tissue. Specifically, the cutting instrument has an elongated body and a cutting wire stored in the elongated body. First, the cutting instrument is placed below the transverse carpal ligament. Next, the cutting wire is fed in the distal direction from the elongated body whereby the cutting wire is made to project in a bow shape to the outside through a window formed in the elongated body. That is, the cutting wire is supported at the distal part and proximal part of the window and deforms so that the intermediate part projects to the outside whereby a bow shaped part is formed. The cutting wire utilizes RF energy to cut the transverse carpal ligament which the cutting wire contacts in the process of projecting out in a bow shape.

### SUMMARY

### Technical Problem

According to this method, a bow-shaped cutting wire is used to cut tissue in the thickness direction, so until the transverse carpal ligament is completely cut, the peak part of the bow-shaped cutting wire damages the outward tissue after passing through the transverse carpal ligament. That is, as shown in FIG. 45A, if trying to cut the target tissue by exactly a predetermined width in the longitudinal direction, since the cutting wire is bow shaped, it is necessary to make the bow shape larger so that the cutting wire extends beyond the thickness of the ligament. This being so, the peak part of the bow-shaped cutting wire passes through the target tissue and ends up damaging the more outward tissue (region D).

Further, as shown in FIG. 45B, it is possible to make the bow shape of the cutting wire smaller than the thickness of the target tissue and make the cutting instrument move back and forth like a saw to cut the target tissue, but this requires additional skill and surgery time. Furthermore, as shown in FIG. 45C, it is possible to make the bow shape of the cutting wire larger than the thickness of the target tissue and to move the cutting instrument toward the target tissue to thereby cut the target tissue all at once. However, as a result, in the state right before cutting, the cutting wire ends up damaging the surrounding tissue (region D).

The present invention has as its object the provision of a less invasive system and method for cutting the target tissue.

### Solution to Problem

According to one aspect of the present invention, there is provided an electrical surgical system for cutting or cauterizing tissue, the electrical surgical system comprising a tubular member having at least one open part and a front end part able to be inserted through the skin and at least one electrode able to cut or cauterize tissue configured to be able to project to the outside of the tubular member and to be able to be retracted to the inside of the tubular member through the at least one open part, in the state where the at least one electrode projects out, a distal end of the at least one electrode being arranged at a position separated from the longitudinal axis of the tubular member.

According to another aspect of the present invention, there is provided a method for cutting or cauterizing tissue, the method comprising making the tubular member pierce a predetermined location, arranging the tubular member near the target tissue, making a distal end of at least one electrode able to cut or able to cauterize tissue and configured to be able to project to the outside of the tubular member and to be able to be retracted to the inside of the tubular member through at least one open part of the tubular member arranged at a position separated from the longitudinal axis of the tubular member, and running high frequency current through the at least one electrode.

In the state where the at least one electrode projects out, the at least one electrode may also extend in the radial direction, the distal direction, or the proximal direction. The at least one open part may also be formed at a side surface of the tubular member. The amount of projection of the at least one electrode can be adjusted. At the inside of the tubular member, a guide part may be formed configured to guide the at least one electrode in a direction toward the at least one open part at the time of projection of the at least one electrode. The tubular member may have a limiting part preventing rotation of the at least one electrode about an axis of the tubular member in the state where the at least one electrode projects out. At least part of the at least one electrode projecting to the outside of the tubular member may be insulated. Graduations may be formed at the outside surface of the tubular member in a longitudinal direction. The at least one open part may be formed so as to enable movement of the at least one electrode along a longitudinal direction of the tubular member in the state where the at least one electrode projects out. An ultrasonic image diagnosis device may be further provided. A plurality of recessed parts or projecting parts may be formed at the outer surface of the tubular member.

### Advantageous Effects of Invention

According to the embodiments of the present invention, the common effect is exhibited of provision of a low invasive system and method for cutting the target tissue.

### BRIEF DESCRIPTION OF DRAWINGS

FIG. 1 is a schematic view of an electrical surgical system for cutting or cauterizing tissue in an embodiment of the present invention.

FIG. 2A is a perspective view of an electrical surgical instrument according to a first embodiment in a stored state of an electrode.

FIG. 2B is a perspective view of an electrical surgical instrument according to the first embodiment in a projected state of an electrode.

FIG. 3 is a perspective view of a counter electrode plate according to a first embodiment.

FIG. 4 is a perspective view of a counter electrode plate according to a second embodiment.

FIG. 5A is a perspective view of a guide needle in a stored state of an electrode.

FIG. 5B is a vertical cross-sectional view of the guide needle in a stored state of an electrode.

FIG. 6A is a perspective view of a guide needle in a projected state of an electrode.

FIG. 6B is a vertical Cross-sectional view of a guide needle in a projected state of an electrode.

FIG. 7A is a schematic view of a state of arrangement of a guide needle near the target tissue.

FIG. 7B is a schematic view of a state of projection of an electrode from the state of FIG. 7A.

FIG. 7C is a schematic view of a state in the middle of making a guide needle move from the state of FIG. 7B to fully cut the target tissue.

FIG. 7D is a schematic view of a state of completion of cutting of the target tissue.

FIG. 8 is a vertical cross-sectional view of a guide needle in an electrical surgical instrument according to the second embodiment.

FIG. 9A is a vertical cross-sectional view of a guide needle in a stored state in an electrical surgical instrument according to a third embodiment.

FIG. 9B is a vertical cross-sectional view of a guide needle in a projected state in an electrical surgical instrument according to the third embodiment.

FIG. 10A is a vertical cross-sectional view of a guide needle in a stored state in an electrical surgical instrument according to a fourth embodiment.

FIG. 10B is a vertical cross-sectional view of a guide needle in a projected state in an electrical surgical instrument according to the fourth embodiment.

FIG. 11A is a vertical cross-sectional view of a guide needle in a stored state in an electrical surgical instrument according to a fifth embodiment.

FIG. 11B is a vertical cross-sectional view of a guide needle in a projected state in an electrical surgical instrument according to the fifth embodiment.

FIG. 12A is a vertical cross-sectional view of a guide needle in a stored state in an electrical surgical instrument according to a sixth embodiment.

FIG. 12B is a vertical cross-sectional view of a guide needle in a projected state in an electrical surgical instrument according to the sixth embodiment.

FIG. 13A is a vertical cross-sectional view of a guide needle in a stored state in an electrical surgical instrument according to a seventh embodiment.

FIG. 13B is a vertical cross-sectional view of a guide needle in a projected state in an electrical surgical instrument according to the seventh embodiment.

FIG. 14A is a vertical cross-sectional view of a guide needle in a stored state in an electrical surgical instrument according to an eighth embodiment.

FIG. 14B is a vertical cross-sectional view of a guide needle in a projected state in an electrical surgical instrument according to the eighth embodiment.

FIG. 15A is a vertical cross-sectional view of a guide needle in a stored state in an electrical surgical instrument according to a ninth embodiment.

FIG. 15B is a vertical cross-sectional view of a guide needle in a projected state in an electrical surgical instrument according to the ninth embodiment.

FIG. 16A is a vertical cross-sectional view of a guide needle in a stored state in an electrical surgical instrument according to a 10th embodiment.

FIG. 16B is a vertical cross-sectional view of a guide needle in a projected state in an electrical surgical instrument according to the 10th embodiment.

FIG. 17A is a vertical cross-sectional view of a guide needle in a stored state in an electrical surgical instrument according to an 11th embodiment.

FIG. 17B is a vertical cross-sectional view of a guide needle in a projected state in an electrical surgical instrument according to the 11th embodiment.

FIG. 18A is a vertical cross-sectional view of a guide needle in a stored state in an electrical surgical instrument according to a 12th embodiment.

FIG. 18B is a vertical cross-sectional view of a guide needle in a projected state in an electrical surgical instrument according to the 12th embodiment.

FIG. 19 is a perspective view of a guide needle in a projected state in an electrical surgical instrument according to a 13th embodiment.

FIG. 20 is a perspective view of a guide needle in a projected state in an electrical surgical instrument according to a 14th embodiment.

FIG. 21 is a front view of a guide needle in a projected state in an electrical surgical instrument according to a 15th embodiment.

FIG. 22 is a vertical cross-sectional view of a guide needle in an electrical surgical instrument according to a 16th embodiment.

FIG. 23 is a vertical cross-sectional view of a guide needle in an electrical surgical instrument according to a 17th embodiment.

FIG. 24A is a vertical cross-sectional view of a guide needle in the stored state in an electrical surgical instrument according to an 18th embodiment.

FIG. 24B is a vertical cross-sectional view of a guide needle in a projected state in an electrical surgical instrument according to the 18th embodiment.

FIG. 24C is an enlarged view of a part A of FIG. 24A.

FIG. 25A is a perspective view of a guide needle in a stored state in an electrical surgical instrument according to a 19th embodiment.

FIG. 25B is a vertical cross-sectional view of a guide needle in the stored state in an electrical surgical instrument according to the 19th embodiment.

FIG. 25C is a perspective view of a guide needle in a projected state in an electrical surgical instrument according to the 19th embodiment.

FIG. 25D is another perspective view of a guide needle in a projected state in an electrical surgical instrument according to the 19th embodiment.

FIG. 26 is a vertical cross-sectional enlarged view of a guide needle in a projected state in an electrical surgical instrument according to a 20th embodiment.

FIG. 27 is a vertical cross-sectional enlarged view of a guide needle in a projected state in an electrical surgical instrument according to a 21st embodiment.

FIG. 28 is a vertical cross-sectional enlarged view of a guide needle in a projected state in an electrical surgical instrument according to a 22nd embodiment.

FIG. 29 is a vertical cross-sectional enlarged view of a guide needle in a projected state in an electrical surgical instrument according to a 23rd embodiment.

FIG. 30 is a vertical cross-sectional enlarged view of a guide needle in a projected state in an electrical surgical instrument according to a 24th embodiment.

FIG. 31 is a vertical cross-sectional enlarged view of a guide needle in a projected state in an electrical surgical instrument according to a 25th embodiment.

FIG. 32 is a perspective view of a guide needle in a projected state in an electrical surgical instrument according to a 26th embodiment.

FIG. 33 is a perspective view of a guide needle in a projected state in an electrical surgical instrument according to a 27th embodiment.

FIG. 34 is a perspective view of a guide needle in a projected state in an electrical surgical instrument according to a 28th embodiment.

FIG. 35 is a perspective view of a guide needle in a projected state in an electrical surgical instrument according to a 29th embodiment.

FIG. 36 is a perspective view of a guide needle in a projected state in an electrical surgical instrument according to a 30th embodiment.

FIG. 37 is a vertical cross-sectional enlarged view of a guide needle in a projected state in an electrical surgical instrument according to a 31st embodiment.

FIG. 38 is a vertical cross-sectional enlarged view of a guide needle in a projected state in an electrical surgical instrument according to a 32nd embodiment.

FIG. 39A is a perspective view of a guide needle in a projected state in an electrical surgical instrument according to a 33rd embodiment.

FIG. 39B is another perspective view of a guide needle in a projected state in an electrical surgical instrument according to the 33rd embodiment.

FIG. 40A is a perspective view of a guide needle in a projected state in an electrical surgical instrument according to a 34th embodiment.

FIG. 40B is another perspective view of a guide needle in a projected state in an electrical surgical instrument according to the 34th embodiment.

FIG. 41A is a perspective view of a guide needle in a projected state in an electrical surgical instrument according to a 35th embodiment.

FIG. 41B is a vertical cross-sectional view of a guide needle in a projected state in an electrical surgical instrument according to the 35th embodiment.

FIG. 42 is a perspective view of a guide needle in a projected state in an electrical surgical instrument according to a 36th embodiment.

FIG. 43 is a vertical cross-sectional view of a guide needle in a projected state in an electrical surgical instrument according to a 37th embodiment.

FIG. 44A is a perspective view of a guide needle in a projected state in an electrical surgical instrument according to a 38th embodiment.

FIG. 44B is another perspective view of a guide needle in a projected state in an electrical surgical instrument according to the 38 embodiment.

FIG. 45A is a schematic view showing a state of cutting of the target tissue by a conventional electrical surgical instrument.

FIG. 45B is another schematic view showing a state of cutting of the target tissue by a conventional electrical surgical instrument.

FIG. 45C is still another schematic view showing a state of cutting of the target tissue by a conventional electrical surgical instrument.

### DESCRIPTION OF EMBODIMENTS

Below, embodiments of the present invention will be explained in detail while referring to the drawings. In the drawings, the corresponding components are assigned common reference notations. Note that the content described below does not limit the technical scope of the inventions described in the claims and the meanings of the terms.

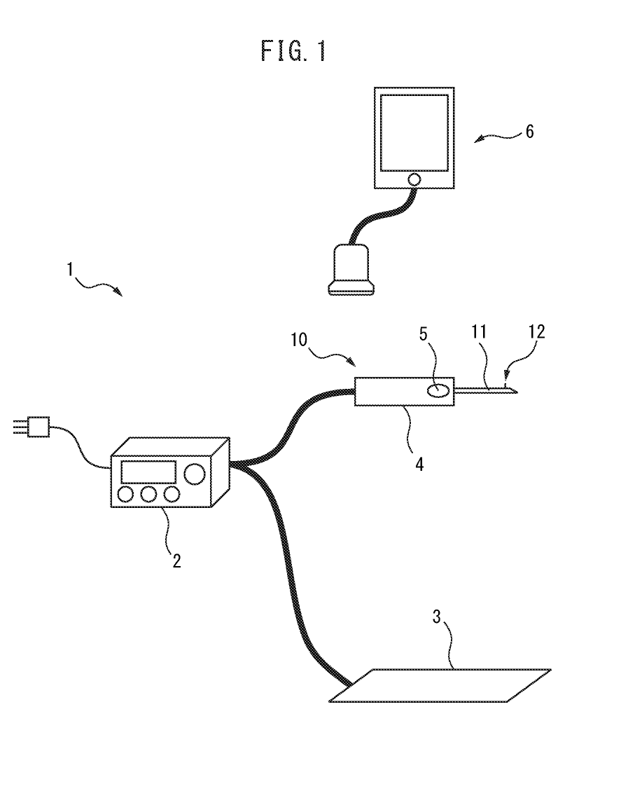

FIG. 1 is a schematic views of an electrical surgical system 1 for cutting or cauterizing tissue according to an embodiment of the present invention. The electrical surgical system 1 has a power supply unit 2, a counter electrode plate 3, and an electrical surgical instrument 10. The electrical surgical instrument 10 has a handle 4, an electrode controller 5, a guide needle 11, and an electrode 12. The power supply unit 2 and electrical surgical instrument 10 are electrically connected, while the power supply unit 2 and the counter electrode plate 3 are electrically connected. The electrical surgical system 1 may also have an ultrasonic image diagnosis apparatus 6.

The principle of cutting or cauterizing tissue by the electrical surgical system 1 is similar to an electrical scalpel or other monopolar type electrical surgical devices using high frequency current. That is, in the electrical surgical system 1, the counter electrode plate 3 is attached so as to contact part of the body of a patient. The surgeon operates the handle 4 while making the electrode 12 contact the target tissue. The instant the electrode 12 contacts the tissue, the high frequency current flows from the power supply unit 2 toward the electrode 12. The high frequency current passes through the body of the patient, then returns through the counter electrode plate 3 to the power supply unit 2. When high frequency current flows, the electrode 12 itself does not generate heat. In the tissue near the high current density electrode 12, Joules heat is generated by the electrical resistance and the tissue is cut or cauterized. That is, the cutting or cauterization of the tissue by this principle utilizes electrical energy. Note that cutting and cauterization will simply referred to together as “cutting”.

The power supply unit 2 is a high frequency transmitter of several hundred kHz to several MHz (for example, 500 kHz). In the power supply unit 2, the maximum output power is for example 200W to 400W. The load resistance is for example 200Ω to 1000Ω.

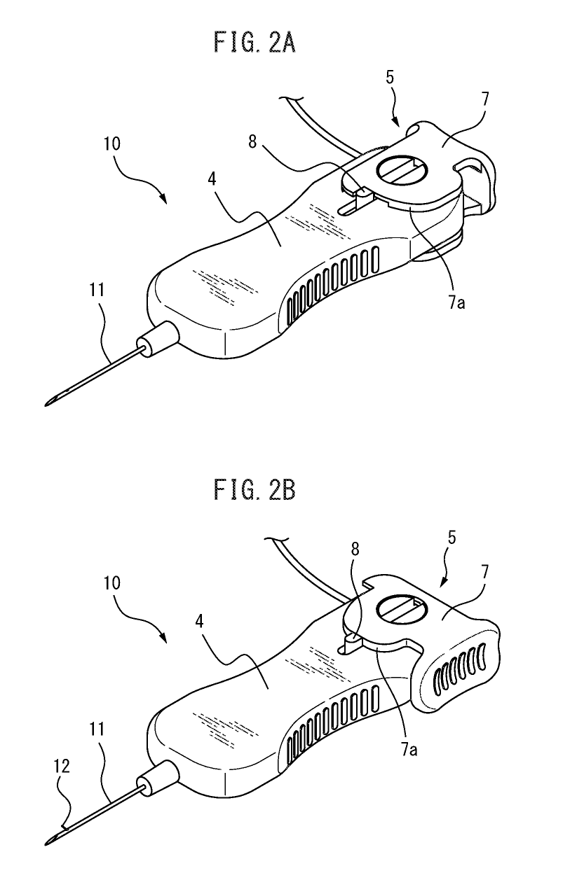

FIG. 2A is a perspective view of an electrical surgical instrument 10 according to a first embodiment in a stored state of the electrode 12, while FIG. 2B is a perspective view of an electrical surgical instrument 10 according to the first embodiment in a projected state of the electrode 12. The electrical surgical instrument 10, as explained above, has a handle 4, an electrode controller 5, a guide needle 11, and an electrode 12. In this embodiment, the electrode controller 5 has a rotating lever 7 and a slide rod 8. Other controller configurations may be possible. The guide needle 11 is a hollow and rigid elongated tubular member. One end of the guide needle 11 is attached to the front end part of the handle 4. The length of the guide needle 11 extends from the handle 4, for example 20 mm to 150 mm. The electrode 12 is a wire-shaped member. The range of the diameter of the electrode 12 is for example 0.1 mm to 0.5 mm. One end of the electrode 12 is connected to the slide rod 8 inside the handle 4, while the other end of the electrode 12 is stored inside the guide needle 11.

The electrode controller 5 consists of the rotating lever 7 and the slide rod 8. The rotating lever 7 has a cam face 7a. The slide rod 8 is biased backward by a not shown elastic member. While making the rotating lever 7 turn, the slide rod 8 receives force from the cam face 7a and slides forward, that is, to the distal direction (FIG. 2B). By the slide rod 8 sliding in the distal direction, the electrode 12 projects out from the guide needle 11 as explained later. If making the rotating lever 7 turn in the reverse direction, the slide rod 8 receives biasing force from the elastic member and is returned to its original position backward, that is, in the proximal direction (FIG. 2A). By the slide rod 8 sliding in the proximal direction, the electrode 12 retracts and is stored in the guide needle 11. Note that, the electrode controller 5 may be configured in any way structurally or electrically so long as it can allow the electrode 12 project out or be stored.

The counter electrode plate 3 may employ any shape or structure so long as configured to not unintentionally detach. For example, FIG. 3 is a perspective view of the counter electrode plate 3 according to a first embodiment, while FIG. 4 is a perspective view of the counter electrode plate 3 according to a second embodiment. The counter electrode plate 3 of FIG. 3 is overall a band shape wound around the arm or leg etc. and can be fastened by a surface fastener etc. The counter electrode plate 3 of FIG. 4 is overall a clip shape for clamping the arm or leg etc. The counter electrode plate 3 may be attached directly to the skin by a seal etc. Further, the counter electrode plate 3 may have flexibility enabling it to be freely bent.

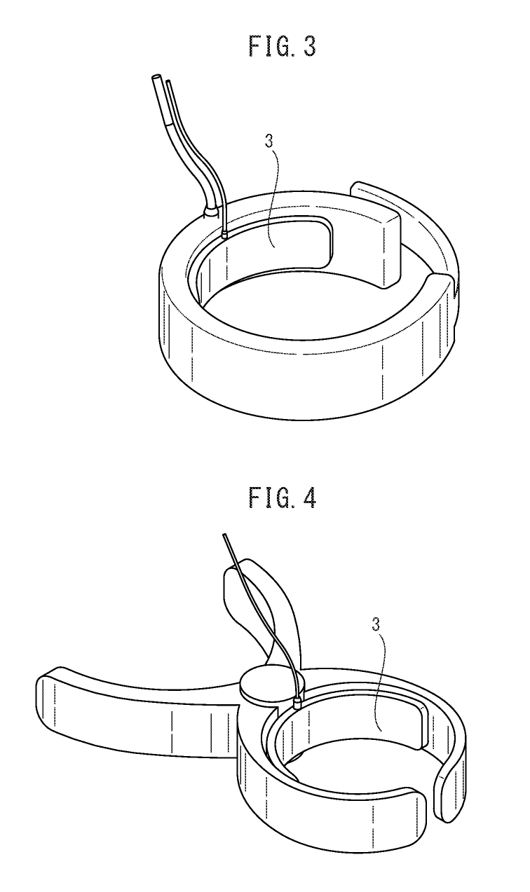

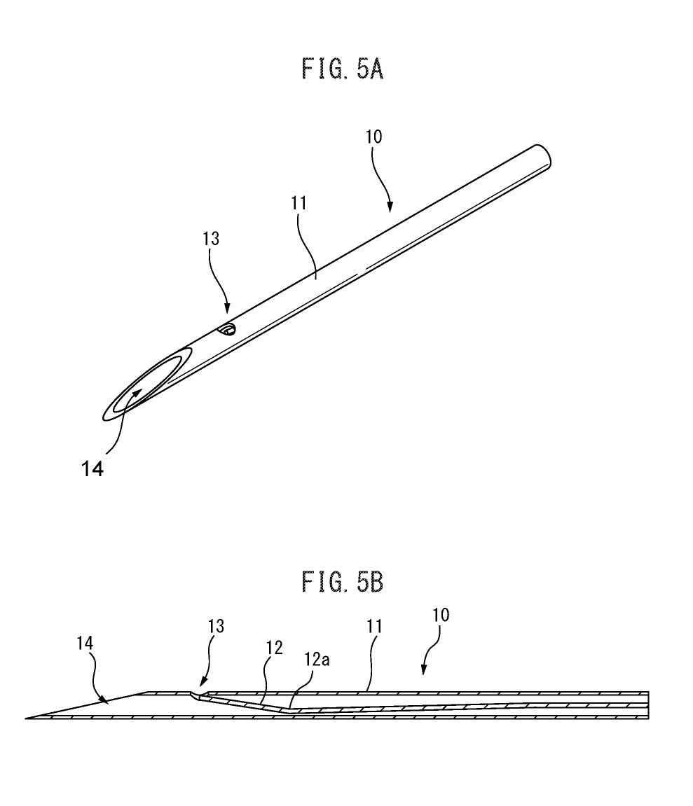

The structure of the guide needle 11 and operation of the electrode 12 will be explained while referring to FIG. 5A and FIG. 5B. FIG. 5A is a perspective view of the guide needle 11 in a stored state of the electrode 12, while FIG. 5B is a vertical cross-sectional view of the guide needle 11 in the stored state of the electrode 12. The guide needle 11, as explained above, is a tubular member. The front end part of the guide needle 11 is slanted to form a sharp edge. Therefore, the front end part of the guide needle 11 can pierce the skin, that is, can be inserted epidermally. At the side surface of the guide needle 11, in particular, the side surface near the front end part, an approximately circular open part 13 is formed. At the front end part of the guide needle 11, another open part comprised of a front end open part 14 is formed. Note that, any shape of the front end part of the guide needle 11 may be employed so long as to enable a piercing action.

As for the guide needle 11, it is possible to use a general hypodermic needle formed by stainless steel etc. The size which can be used is 16G (gauge) to 25G (outside diameter 0.5 mm to 1.6 mm), preferably 18G to 23 (outside diameter 0.6 mm to 1.2 mm) in range, but a thicker diameter or thinner diameter hypodermic needle may also be used. In this case, the diameter of the open part 13 is preferably a size enabling the electrode 12 to smoothly project out and be stored and able to prevent the electrode 12 from rotating about the axis of the guide needle 11. For example, it is 0.5 mm to 1.2 mm in range.

The electrode 12 is stored inside the guide needle 11 in the state before being made to project out from the guide needle 11. The electrode 12, for example, is a single wire or twisted wire of stainless steel, but may also be a single wire of tungsten. The electrode 12 may also be formed from another material and preferably has a suitable rigidity, elasticity, and biocompatibility. The diameter of the electrode 12 is, for example, 0.2 mm. The front end of the electrode 12, that is, the distal end, is oriented toward the open part 13 in the state stored at the inside of the guide needle 11. The electrode 12, for example, as shown in FIG. 5B, is oriented toward the open part 13 by having an obtuse angle at bent part 12a.

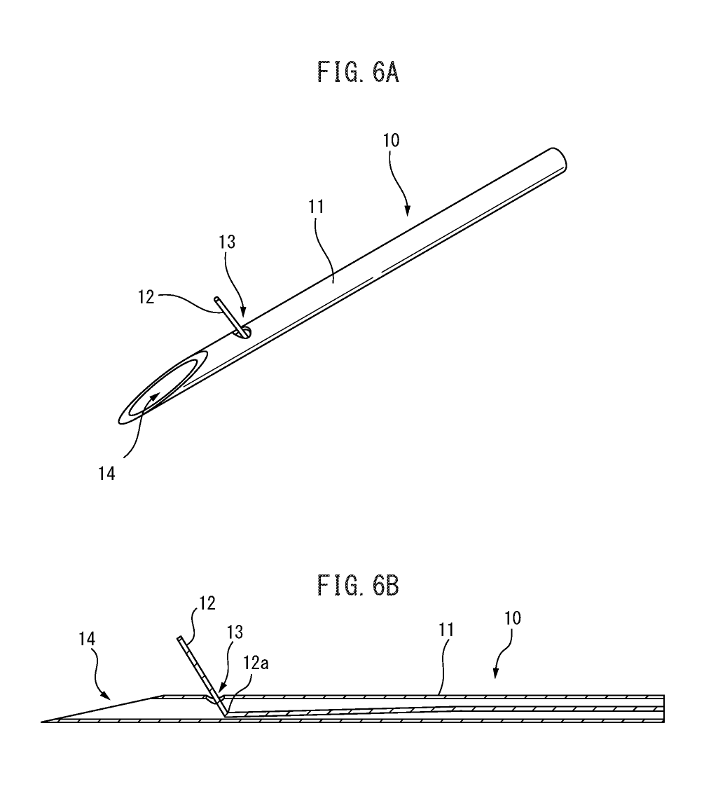

FIG. 6A is a perspective view of the guide needle 11 in a projected state of the electrode 12, while FIG. 6B is a vertical cross-sectional view of the guide needle 11 in the projected state of the electrode 12. If operating the electrode controller 5, that is, if making the rotating lever 7 turn, the slide rod 8 slides in the distal direction whereby the electrode 12 connected to the slide rod 8 also slides in the distal direction with respect to the guide needle 11. The electrode 12 bent from the bent part 12a while sliding in the distal direction. As a result, the distal end of the electrode 12 projects to the outside of the guide needle 11 through the open part 13 of the guide needle 11 (FIG. 6A and. FIG. 6B). The electrode 12 extends for example up to 20 mm radially from the outside of the guide needle 11.

In more detail, as shown in FIG. 6A and FIG. 6B, in the state where the electrode 12 is projected, the distal end of the electrode 12 is arranged at a position separated from the longitudinal axis of the guide needle 11. In other words, the distal end of the electrode 12 projects out in the radial direction, that is, has a radial direction component, and is supported by the guide needle 11 in a cantilever manner. Therefore, the distal end of the electrode 12 extends in the distal direction and is arranged in a region not a space which the outside shape of the guide needle 11 creates on an extension in the longitudinal direction.

If again operating the electrode controller 5, that is, if making the rotating lever 7 turn in the reverse direction, the slide rod 8 slides in the proximal direction. The electrode 12 connected to the slide rod 8 also slides in the proximal direction with respect to the guide needle 11. As a result, the electrode 12 retracts and is again stored in the guide needle 11 (FIG. 5A and FIG. 5B).

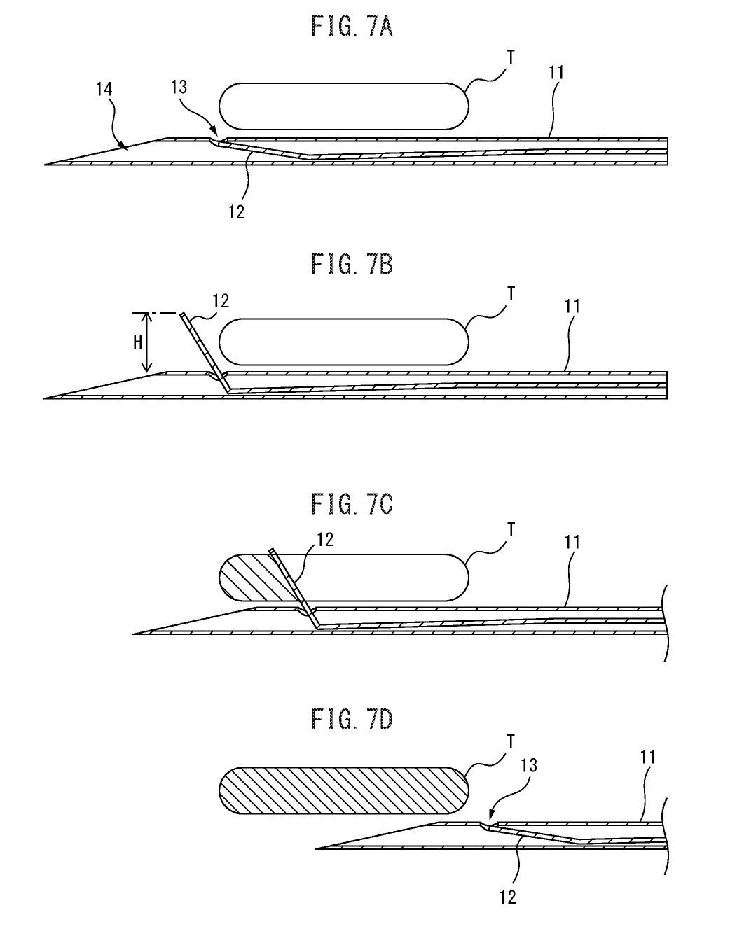

While referring to FIG. 7A to FIG. 7D, the method of cutting tissue using the electrical surgical system 1 will be explained. FIG. 7A is a schematic view of the state of arrangement of the guide needle 11 near the target tissue T, FIG. 7B is a schematic view of the state of projection of the electrode 12 from the state of FIG. 7A, FIG. 7C is a schematic view of the state in the middle of making the guide needle 11 move from the state of FIG. 7B to cut the target tissue T, and FIG. 7D is a schematic view of the state of completion of cutting of the target tissue. The target tissue T in the figures is a ligament or tendon. Ligaments and tendons are flat and strong soft tissue and are suitable for cutting using the electrical surgical system 1. The electrical surgical system 1 may also be used for tissue other than ligaments and tendons, for example, nerves, veins, and tumors.

First, the guide needle 11 of the electrical surgical instrument 10 is inserted epidermally and the guide needle 11 pushed in until the guide needle 11 is arranged near the target tissue T. At this time, the guide needle 11 is arranged so as to become parallel to flat tissue and so as to become parallel to the cutting direction. Further, the guide needle 11 is inserted along the cutting direction until arranged at a position where the open part 13 exceeds the target tissue T (FIG. 7A). Next, the electrode controller 5 is operated to make the electrode 12 project out from the open part 13 of the guide needle 11 (FIG. 7B). At this time, the amount of projection of the electrode 12 is set to an extent exceeding the height of the target tissue T in the distance H in the radial direction. Then, pulling the guide needle 11 in the proximal direction while running high frequency current through the electrode 12 will cut the target tissue T (FIG. 7C). When the target tissue T finishes being cut, the high frequency current is stopped and the electrode controller 5 is operated to store the electrode 12 to the guide needle 11 (FIG. 7D). Finally, the guide needle 11 is completely pulled out from the body whereupon the treatment is ended.

To confirm the position and posture of the guide needle 11, the amount of projection of the electrode 12, the cut state of the target tissue T, etc., an ultrasonic image diagnosis device 6 may be used. Further, a high frequency current may be run linked with the operation of the electrode controller 5 through rotation of the rotating lever 7. That is, if making the rotating lever 7 turn to make the electrode 12 project out, high frequency current flows. If making the rotating lever 7 turn in the reverse direction and storing the electrode 12, the high frequency current is stopped. Note that, it is also possible to provide another switch, for example, a foot switch able to be operated by the foot, and control the start and stopping of the high frequency current. It is also possible to use a voice command of the surgeon to control the start and stopping of the high frequency current.

Taking as an example surgery for carpal tunnel syndrome, a more specific method will be explained. The surgeon attaches the counter electrode plate 3 to part of the body of the patient and if necessary, provide anesthesia. Next, the surgeon inserts the guide needle 11 from the wrist side of the patient to directly under the transverse carpal ligament while viewing the procedure by the ultrasonic image diagnosis device 6. When the open part 13 of the guide needle 11 reaches the distal side end part of the transverse carpal ligament, insertion is stopped and the electrode 12 is made to project out. Next, a high frequency current is run through the electrode 12 and the guide needle 11 is pulled while cutting the transverse carpal ligament. The ultrasonic image diagnosis device 6 is used to confirm the cut, the high frequency current is turned off, the electrode 12 is returned to the stored state and the guide needle 11 is pulled out from the wrist. Finally, an appropriate patch is placed over the treated part and the counter electrode plate 3 is removed thereby ending the surgery.

The electrical surgical system 1 can be applied to, in addition to carpal tunnel syndrome, arthroscopic surgery and other electrical surgery for surgical and dental use. As specific examples able to be applied to, plantar fascia dissection, tarsal tunnel release surgery, cubital tunnel release surgery, gastrocnemius release surgery, nerve cautery, vascular occlusion surgery, excision and cauterization of tumors, varicose vein ablation, adhesion resection, hernia surgery, spinal canal surgery, aortic valve replacement, etc. are suggested, but the invention is not limited to these. The electrical surgical system 1 can also be applied to animals in addition to humans.

According to the electrical surgical system 1, by inserting the guide needle 11 epidermally, it approaches the target tissue T, so compared with cutting open the skin, treatment is possible with low invasiveness. Further, by suitably adjusting the amount of projection of the electrode 12 in accordance with the thickness of the target tissue T, it is possible to keep the damage to the surrounding tissue to a minimum. Further, when making the electrode 12 project out, high frequency current is not run, so it is possible to minimize damage to the tissue while making the electrode 12 project out. Further, in the state where the electrode 12 projects out, the distal end of the electrode 12 is arranged at a position separate from the axis of the guide needle 11, so it is possible to keep the damage to the surrounding tissue to a minimum. Further, a single pullout operation ends the cutting of the target tissue T, so it is possible to keep the damage to the surrounding tissue to a minimum.

Below, embodiments other than the electrical system 1 exhibiting effects similar to these effects will be explained.

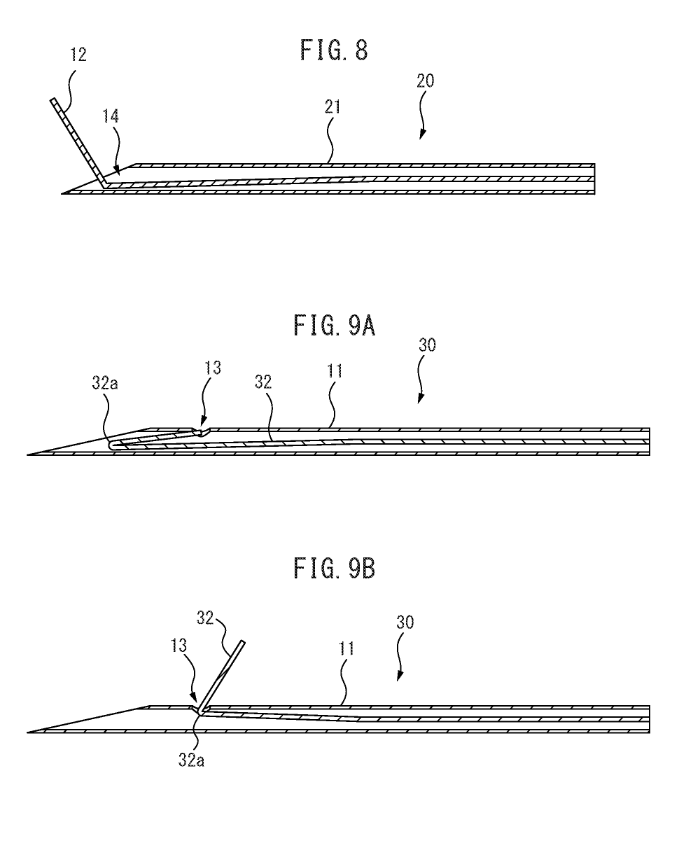

FIG. 8 is a vertical cross-sectional view of a guide needle 21 in an electrical surgical instrument 20 according to the second embodiment. The guide needle 21, compared with the guide needle 11 according to the above-mentioned first embodiment, does not have an open part 13. Instead, the electrode 12 is made to project out or be stored through the distal open end part 14.

FIG. 9A is a vertical cross-sectional view of a guide needle 11 in the stored state in an electrical surgical instrument 30 according to a third embodiment, while FIG. 9B is a vertical cross-sectional view of the guide needle 11 in the projected state in the electrical surgical instrument 30 according to the third embodiment. In the present embodiment, the electrode 32 is stored in the guide needle 11 so as to have an acute angle at the bent part 32a. As a result, in the projected state, the distal end of the electrode 32 extends in the proximal direction. By the distal end of the electrode 32 extending in the proximal direction, when pulling the guide needle 11 in the proximal direction to cut the target tissue T, it is possible to catch the target tissue T between the guide needle 11 and the electrode 32 and reliably cut it.

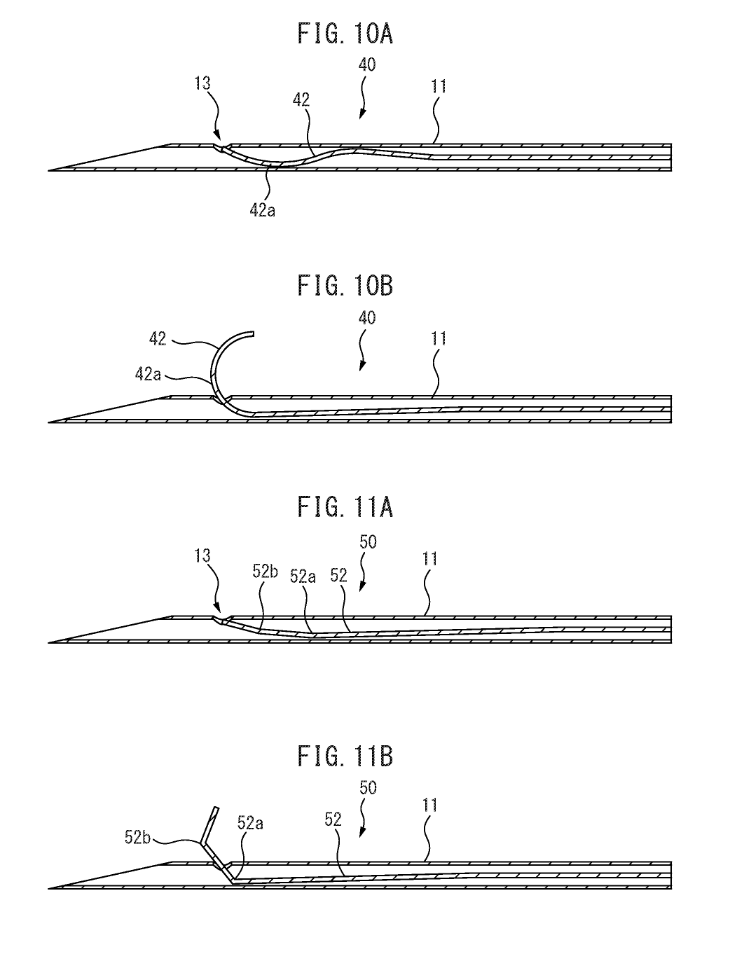

FIG. 10A is a vertical cross-sectional view of a guide needle 11 in a stored state in an electrical surgical instrument 40 according to a fourth embodiment, while FIG. 10B is a vertical cross-sectional view of the guide needle 11 in a projected state in the electrical surgical instrument 40 according to the fourth embodiment. In the present embodiment, the electrode 42 has a curved part 42a. As a result, in the projected state, the distal end of the electrode 42 extends in the proximal direction. By the distal end of the electrode 42 extending in the proximal direction, when pulling the guide needle 11 in the proximal direction to cut the target tissue T, it is possible to catch the target tissue T between the guide needle 11 and the electrode 42 and reliably cut it.

FIG. 11A is a vertical cross-sectional view of a guide needle 11 in the stored state in an electrical surgical instrument 50 according to a fifth embodiment, while FIG. 11B is a vertical cross-sectional view of the guide needle 11 in a projected state in the electrical surgical instrument 50 according to the fifth embodiment. In the present embodiment, the electrode 52 has an obtuse angle in a first bent part 52a and an obtuse angle in a second bent part 52b arranged in the more distal section of electrode 52. The first bent part 52a and the second bent part 52b are bent in the same direction. As a result, in the projected state, the distal end of the electrode 52 extends in the proximal direction. By the distal end of the electrode 52 extending in the proximal direction, when pulling the guide needle 11 in the proximal direction to cut the target tissue T, it is possible to catch the target tissue T between the guide needle 11 and the electrode 52 and reliably cut it.

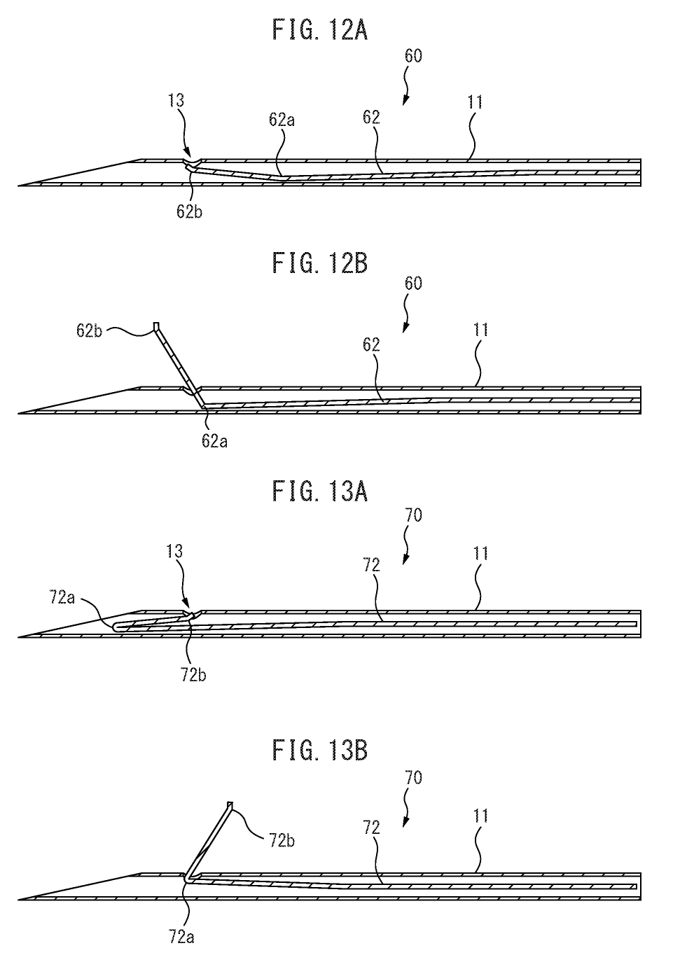

FIG. 12A is a vertical cross-sectional view of a guide needle 11 in a stored state in an electrical surgical instrument 60 according to a sixth embodiment, while FIG. 12B is a vertical cross-sectional view of the guide needle 11 in a projected state in the electrical surgical instrument 60 according to the sixth embodiment. In the present embodiment, the electrode 62 has a first bent part 62a corresponding to the bent part 12a of the electrode 12 of the first embodiment and a second bent part 62b near the distal end. The second bent part 62b, in the stored state, is bent so as to face the open part 13. By adding the second bent part 62b near the distal end, it becomes easy to make the distal end of the electrode 62 project out from the open part 13 of the guide needle 11.

FIG. 13A is a vertical cross-sectional view of a guide needle 11 in the stored state in an electrical surgical instrument 70 according to a seventh embodiment, while FIG. 13B is a vertical cross-sectional view of the guide needle 11 in a projected state in the electrical surgical instrument 70 according to the seventh embodiment. In the present embodiment, the electrode 72 has a first bent part 72a similar to the bent part 32a of the electrode 32 of the third embodiment and a second bent part 72b near the distal end. The second bent part 72b, in the stored state, is bent so as to face the open part 13. By the second bent part 72b being provided near the distal end, it becomes easy to make the distal end of the electrode 72 project out from the open part 13 of the guide needle 11.

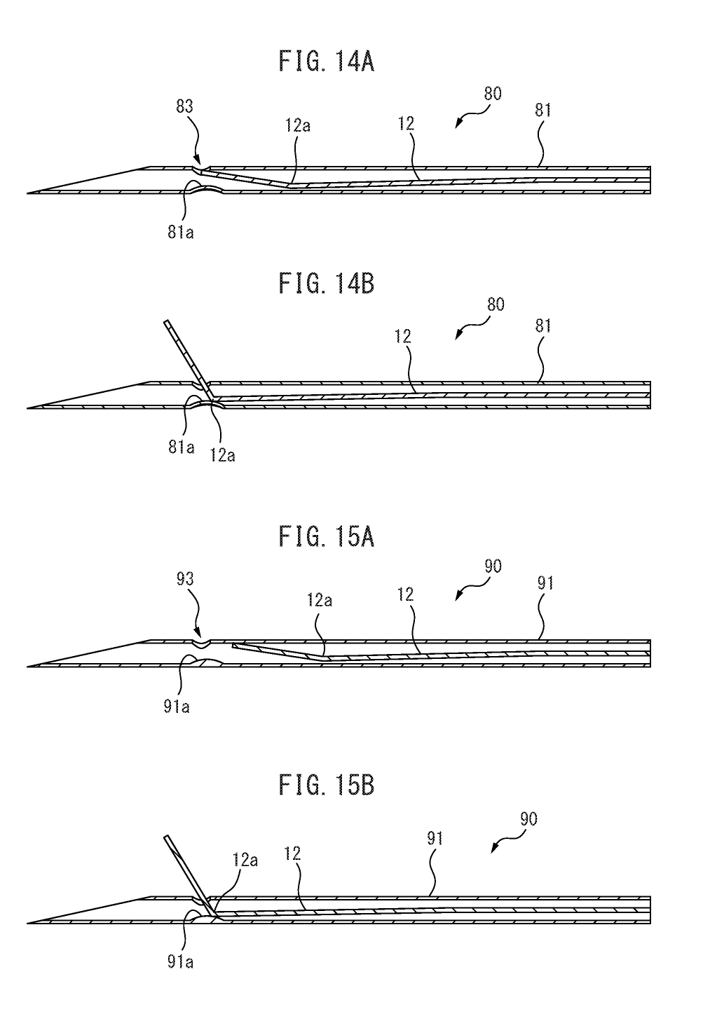

FIG. 14A is a vertical cross-sectional view of a guide needle 81 in a stored state in an electrical surgical instrument 80 according to an eighth embodiment, while FIG. 14B is a vertical cross-sectional view of the guide needle 81 in a projected state in the electrical surgical instrument 80 according to the eighth embodiment. In the present embodiment, at the outside surface of the guide needle 81, an approximately circular open part 83 is formed. Furthermore, at the inside of the guide needle 61, a guide projection 81a is formed facing the open part 83. The guide projection 81a is a guide part and is formed by pressing the outside of the guide needle 81 to the inside in the radial direction. By having a guide projection 81a at the inside surface of the guide needle 81, at the time of projection of the electrode 12, the distal end of the electrode 12 abuts against the guide projection 81a and is guided in a direction toward the open part 83 whereby it becomes easier to make the distal end of the electrode 12 project to the outside from the open part 83 of the guide needle 81.

FIG. 15A is a vertical cross-sectional view of a guide needle 91 in the stored state in an electrical surgical instrument 90 according to a ninth embodiment, while FIG. 15B is a vertical cross-sectional view of the guide needle 91 in a projected state in the electrical surgical instrument 90 according to the ninth embodiment. In the present embodiment, at the side surface of the guide needle 91, an approximately circular open part 93 is formed. Furthermore, at the inside of the guide needle 91, a guide projection 91a is formed facing the open part 93. The guide projection 91a is a guide part and is formed by making that part thicker. By having a guide projection 91a formed at the inside surface of the guide needle 91, at the time of projection of the electrode 12, the distal end of the electrode 12 abuts against the guide projection 91a and is guided in a direction toward the open part 93 whereby it becomes easier to make the distal end of the electrode 12 project to the outside from the open part 93 of the guide needle 91.

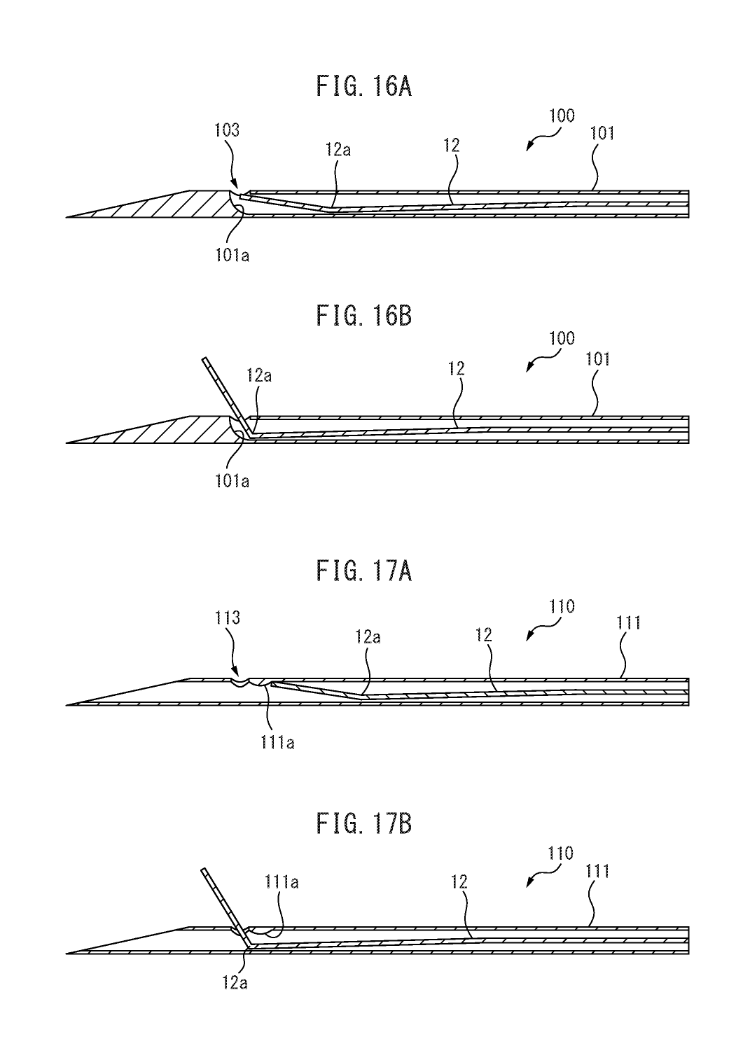

FIG. 16A is a vertical cross-sectional view of a guide needle 101 in the stored state in an electrical surgical instrument 100 according to a 10th embodiment, while FIG. 16B is a vertical cross-sectional view of the guide needle 101 in a projected state in the electrical surgical instrument 100 according to the 10th embodiment. In the present embodiment, at the side surface of the guide needle 101, an approximately circular open part 103 is formed. Furthermore, at the inside of the guide needle 101, a guide part comprised of a guide curved surface 101a is formed. By having the guide curved surface 101a formed at the inside surface of the guide needle 101, at the time of projection of the electrode 12, the distal end of the electrode 12 abuts against the guide curved surface 101a and is guided in a direction toward the open part 103 whereby it becomes easier to make the distal end of the electrode 12 project to the outside from the open part 103 of the guide needle 101.

FIG. 17A is a vertical cross-sectional view of a guide needle 111 in a stored state in an electrical surgical instrument 110 according to an 11th embodiment, while FIG. 17B is a vertical cross-sectional view of the guide needle 111 in a projected state in the electrical surgical instrument 110 according to the 11th embodiment. In the present embodiment, at the side surface of the guide needle 111, an approximately circular open part 113 is formed. Furthermore, at the inside of the guide needle 111, near the open part 113, that is, at the proximal side of the open part 113, the limiting part comprised of a limiting projection 111a is formed. By a limiting projection 111a being formed at the inside surface of the guide needle 111, in the stored state, it is possible to stop the distal end of the electrode 12 by a limiting projection 111a and stably limit the position of the electrode 12.

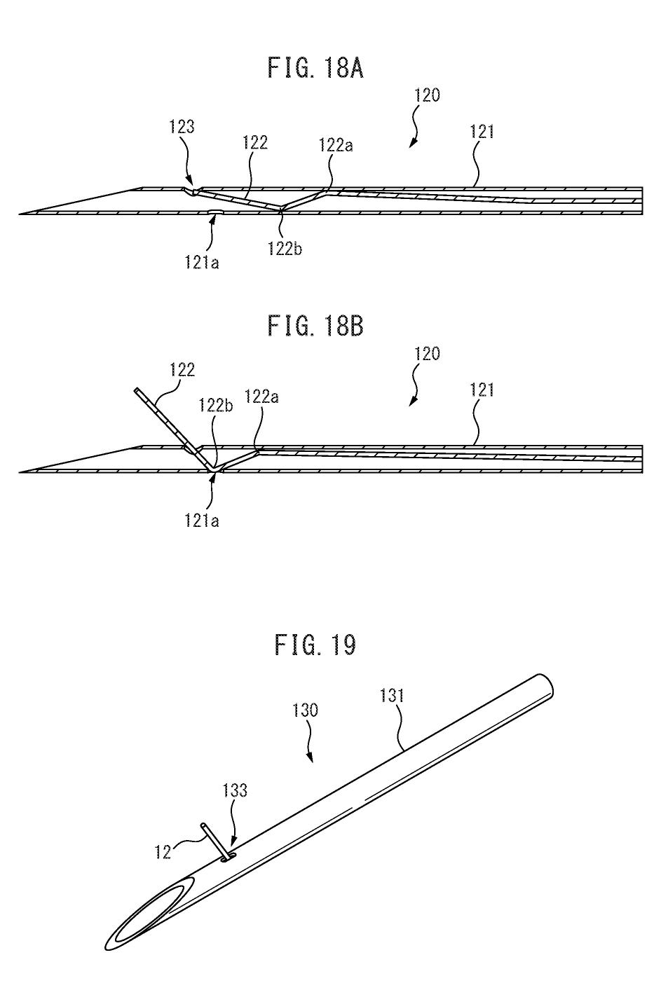

FIG. 18A is a vertical cross-sectional views of a guide needle 121 in a stored state in an electrical surgical instrument 120 according to a 12th embodiment, while FIG. 18B is a vertical cross-sectional view of the guide needle 121 in a projected state in the electrical surgical instrument 120 according to the 12th embodiment. In the present embodiment, at the side surface of the guide needle 121, an approximately circular open part 123 is formed. Furthermore, at the inside of the guide needle 121, a limiting part comprised of a limiting recess 121a is formed in the inside surface facing the open part 123 just slightly in the proximal direction. Further, the electrode 122 has a first bent part 122a and a second bent part 122b at the more distal side. The first bent part 122a and the second bent part 122b are bent in different directions. By the limiting recess 121a being formed at the inside surface of the guide needle 121, in the projected state, the second bent part 122b of the electrode 12 is stopped at the limiting recess 121a and can stably limit the position of the electrode 12. Note that, the limiting recess may also be an opening.

FIG. 19 is a perspective view of a guide needle 131 in a projected state in an electrical surgical instrument 130 according to a 13th embodiment. In the present embodiment, at the side surface of the guide needle 131, an elongated open part 133 extending in the longitudinal direction is formed. The open part 133 of the present embodiment is formed narrower in width in the circumferential direction than the open part of the above-mentioned embodiment. For this reason, the elongated open part 133 performs the role of a limiting part whereby, in the projected state, rotation of the electrode 12 about the axis of the guide needle 131 is prevented.

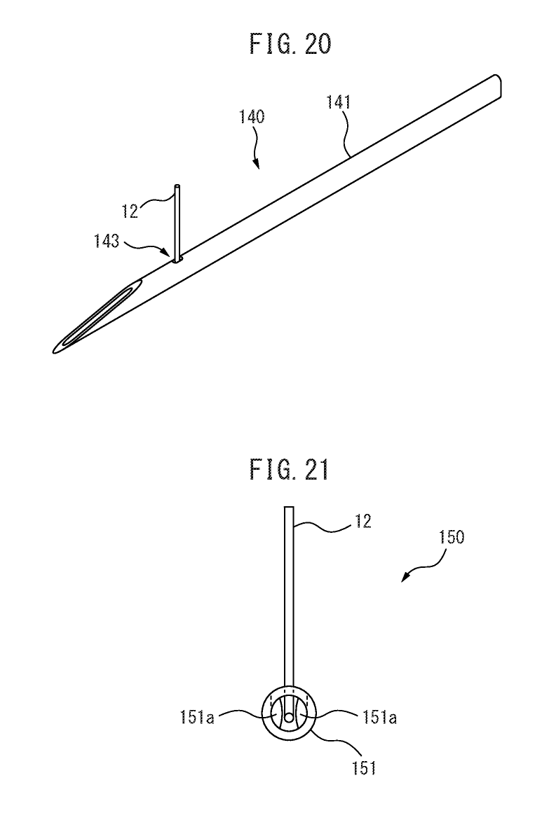

FIG. 20 is a perspective view of a guide needle 141 in a projected state in an electrical surgical instrument 140 according to a 14th embodiment. In the present embodiment, at the side surface of the guide needle 141, an open part 143 is formed. The guide needle 141 is a tubular member having not a circular cross-section, but an elongated cross-sectional shape along the direction of projection of the electrode 12, for example, an oval shape. As a result, at the inside surface of the guide needle 141, there is a small clearance in the direction of intersection of the longitudinal direction of the guide needle 141 and the direction of projection of the electrode 12. The small clearance performs the role of a limiting part. Therefore, in the projected state, rotation of the electrode 12 about the axis of the guide needle 141 is prevented.

FIG. 21 is a front view of a guide needle 151 in a projected state in an electrical surgical instrument 150 according to a 15th embodiment. In the present embodiment, the guide needle 151 is formed with a shape of the inside surface similar to the guide needle 141 of the 11th embodiment. That is, the inside surface of the guide needle 151 is made thicker to form the limiting part comprised of the limiting wall 151a. Therefore, in the projected state, rotation of the electrode 12 about the axis of the guide needle 151 is prevented. Note that, it is also possible to insert another member inside the guide needle to form a limiting wall similar to the limiting wall 151a.

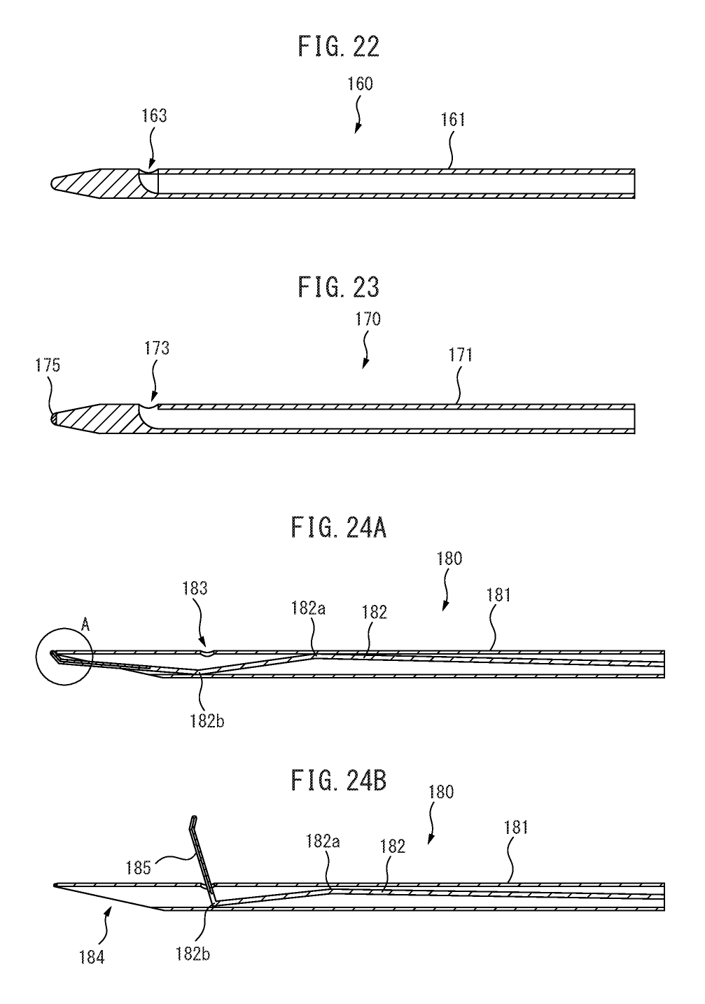

FIG. 22 is a vertical cross-sectional view of a guide needle 161 in an electrical surgical instrument 160 according to a 16th embodiment. In the present embodiment, at the side surface of the guide needle 161, an approximately circular open part 163 is formed. The guide needle 161 has a guide curved surface 161a in the same way as the guide needle 101 in the projected state at the electrical surgical instrument 100 according to the 10th embodiment shown in FIG. 16A and FIG. 16B. Furthermore, the front end of the guide needle 161, that is, the distal end, is formed into a conical shape. The peak point of this conical shape is rounded. By the distal end of the guide needle 161 being rounded, in the middle of insertion of the guide needle 161, it becomes possible to insert it to the target position without getting caught at the boundary part of different tissues.

FIG. 23 is a vertical cross-sectional view of a guide needle 171 in an electrical surgical instrument 170 according to a 17th embodiment. In the present embodiment, at the side surface of the guide needle 171, an approximately circular open part 173 is formed. The guide needle 171 has a similar shape as the guide needle 161 in the electrical surgical instrument 160 according to the 16th embodiment shown in FIG. 22. At the distal end of the guide needle 171, an additional electrode comprised of the auxiliary electrode 175 is provided. By running a high frequency current through the auxiliary electrode 175, it is possible to assist the insertion of the guide needle 171 or use the guide needle 171 itself to cut or cauterize the tissue. Further, it is also possible to provide a resistor or other heating element instead of the auxiliary electrode 175 and use the heat energy to cauterize the tissue or assist the insertion of the guide needle 171.

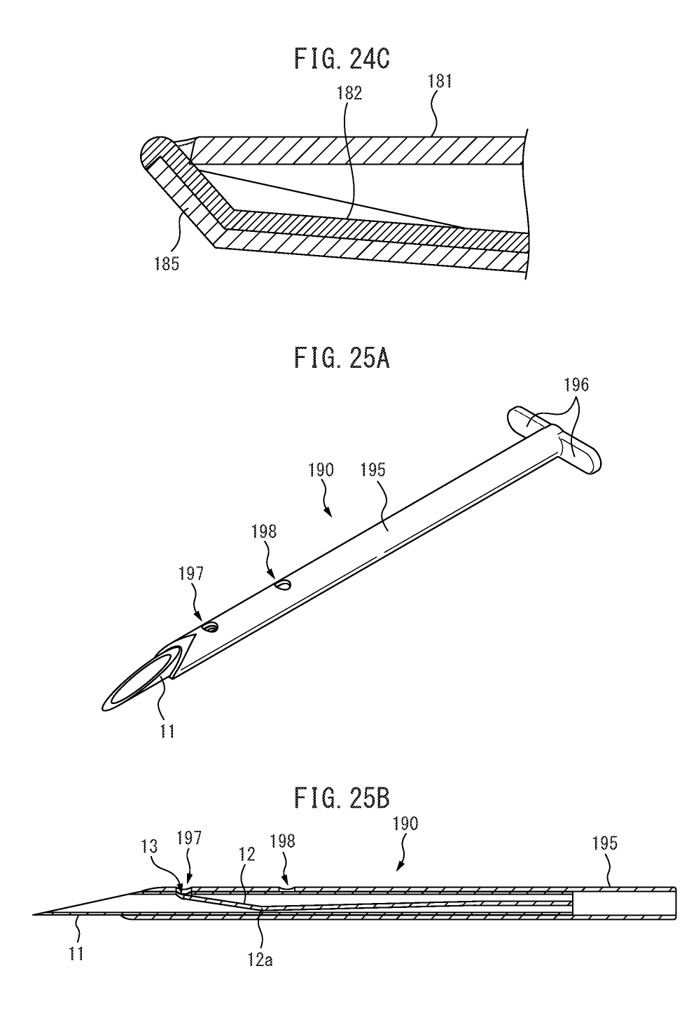

FIG. 24A is a vertical cross-sectional view of a guide needle 181 in a stored state in an electrical surgical instrument 180 according to an 18th embodiment, FIG. 24B is a vertical cross-sectional view of the guide needle 181 in a projected state in the electrical surgical instrument 180 according to the 18th embodiment, and FIG. 24C is an enlarged view of a part A of FIG. 24A. In the present embodiment, at the side surface of the guide needle 181, an approximately circular open part 183 is formed. The front end part of the guide needle 181 is formed with another open part comprised of the front end open part 184. The electrode 182 has an obtuse angle first bent part 182a and an obtuse angle second bent part 182b arranged in the more distal direction. The first bent part 182a and the second bent part 182b are bent in different directions.

The distal end of the electrode 182 is arranged so as to project out to the front just slightly from the distal end of the guide needle 181 (FIG. 24C). Further, the outside surface of the electrode 182, that is, the surface at the outside in the radial direction in the stored state (FIG. 24A) or the surface at the distal side in the projected state (FIG. 24B), is covered by an insulator 185. However, as shown in FIG. 24G, the distal end of the electrode 182 is not covered by the insulator 185 so as to perform the same role as the assisting electrode 175 of the guide needle 171 in the electrical surgical instrument 170 according to the 17th embodiment shown in FIG. 23. In other words, the electrode 182 is covered by the insulator 185 at least in part. Therefore, at the time of insertion of the guide needle 181, it is possible to run a high frequency current through the electrode 182 to assist the insertion of the guide needle 181.

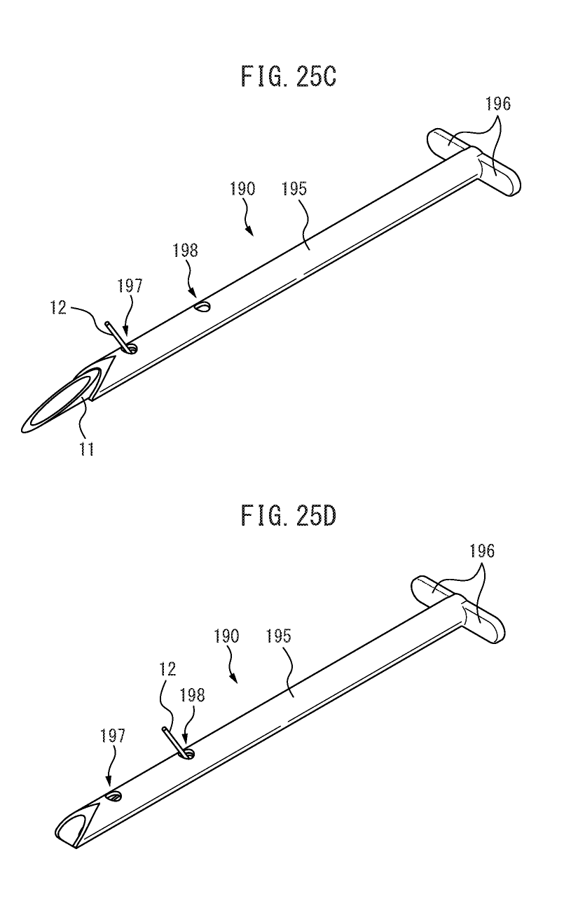

FIG. 25A is a perspective view of a guide needle 11 in a stored state in an electrical surgical instrument 190 according to a 19th embodiment, FIG. 25B is a vertical cross-sectional view of the guide needle 11 in the stored state in the electrical surgical instrument 190 according to the 19th embodiment, FIG. 25C is a per view of the guide needle 11 in a projected state in the electrical surgical instrument 190 according to the 19th embodiment, and FIG. 25D is another per view of the guide needle 11 in a projected state in the electrical surgical instrument 190 according to the 19th embodiment. In the present embodiment, around the guide needle 11, a tubular member comprised of a sleeve member 195 is attached. The sleeve member 195 has an operating part 196 extending in the radial direction. The sleeve member 195 can be made to slide in the axial direction with respect to the guide needle 11 by gripping the operating part 196. Further, at the side surface of the sleeve member 195, the first open part 197 and the second open part 198 at the more proximal side are formed.

By making the sleeve member 195 slide in the proximal direction, the distal end of the guide needle 11 projects out and can pierce the tissue. Further, in this state, it is possible to make the electrode 12 project out through the first open part 197 (FIG. 25C). Further, by making the sleeve member 195 slide in the distal direction, the distal end of the guide needle 11 is stored in the sleeve member 195. By establishing this state, damage to the tissue by the guide needle 11 is prevented. Furthermore, in this state, it is possible to make the electrode 12 project out through the second open part 198 (FIG. 25D).

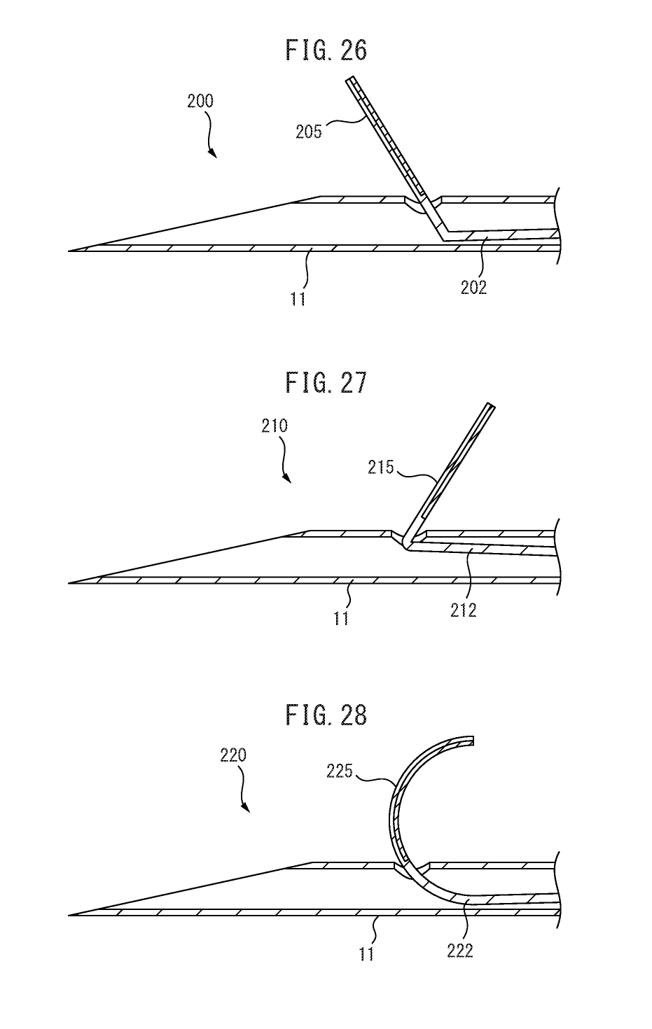

FIG. 26 is a vertical cross-sectional enlarged view of a guide needle 11 in a projected state in an electrical surgical instrument 200 according to a 20th embodiment. The guide needle 11 and electrode 202 of the present embodiment are the same in basic configuration as the guide needle 11 and electrode 12 in the electrical surgical instrument 10 according to the first embodiment. However, the electrode 202 is covered by the insulator 205 at the surface at the distal side in the projected state.

FIG. 27 is a vertical cross-sectional enlarged view of a guide needle 11 in a projected state in an electrical surgical instrument 210 according to a 21st embodiment. The guide needle 11 and electrode 212 of the present embodiment are the same in basic configuration as the guide needle 11 and electrode 32 in the stored state in the electrical surgical instrument 30 according to the third embodiment. However, the electrode 212 is covered by the insulator 215 at the surface at the distal side in the projected state.

FIG. 28 is a vertical cross-sectional enlarged view of a guide needle 11 in a projected state in an electrical surgical instrument 220 according to a 22nd embodiment. The guide needle 11 and electrode 222 of the present embodiment are the same in basic configuration as the guide needle 11 and electrode 42 in the stored state in the electrical surgical instrument 40 according to the fourth embodiment. However, the electrode 222 is covered by the insulator 225 at the surface at the distal side in the projected state.

The electrode 202 of the 20th embodiment, the electrode 212 of the 21st embodiment, and the electrode 222 of the 22nd embodiment are respectively covered by insulators at least at parts of the surfaces at the distal sides in the projected state, so damage to the surrounding tissue is prevented without affecting the efficient cutting of the target tissue T.

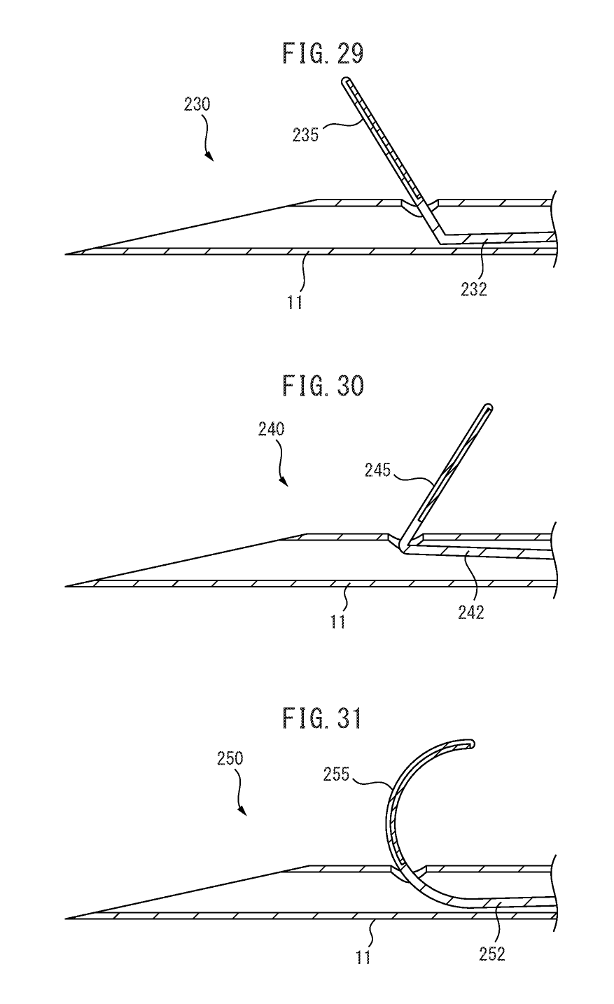

FIG. 29 is a vertical cross-sectional enlarged view of a guide needle 11 in a projected state in an electrical surgical instrument 230 according to a 23rd embodiment, FIG. 30 is a vertical cross-sectional enlarged view of a guide needle 11 in a projected state in an electrical surgical instrument 240 according to a 24th embodiment, and FIG. 31 is a vertical cross-sectional enlarged view of a guide needle 11 in a projected state in an electrical surgical instrument 250 according to a 25th embodiment. These are respectively configured by the electrode 202 of the 20th embodiment, the electrode 212 of the 21st embodiment, and the electrode 222 of the 22nd embodiment with distal ends rounded and covered by insulators. That is, the electrode 232 of the 23rd embodiment is covered by the insulator 235, the electrode 242 of the 24th embodiment is covered by the insulator 245, and the electrode 252 of the 25th embodiment is covered by the insulator 255. Due to this, damage to the surrounding tissue can be further prevented.

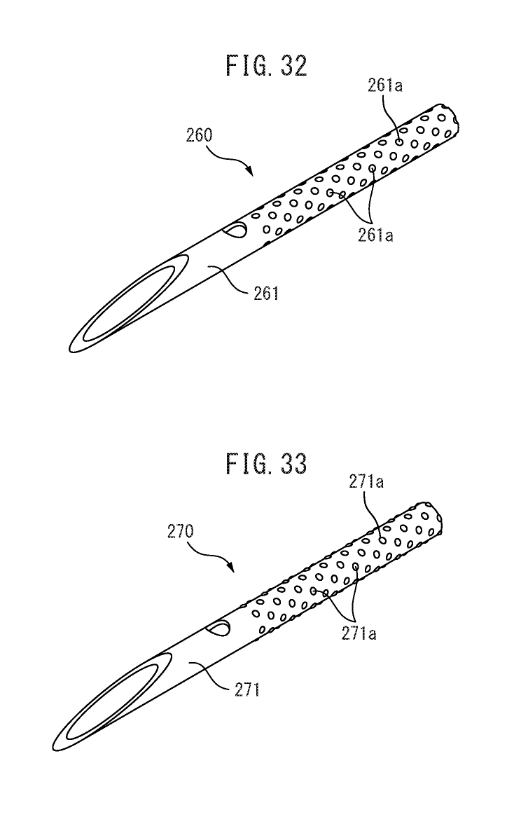

FIG. 32 is a perspective view of a guide needle 261 in a projected state in an electrical surgical instrument 260 according to a 26th embodiment. The surface of the guide needle 261 formed with a plurality of dimples 261a.

FIG. 33 is a perspective view of a guide needle 271 in a projected state in an electrical surgical instrument 270 according to a 27th embodiment. The surface of the guide needle 271 formed with a plurality of projections 271a.

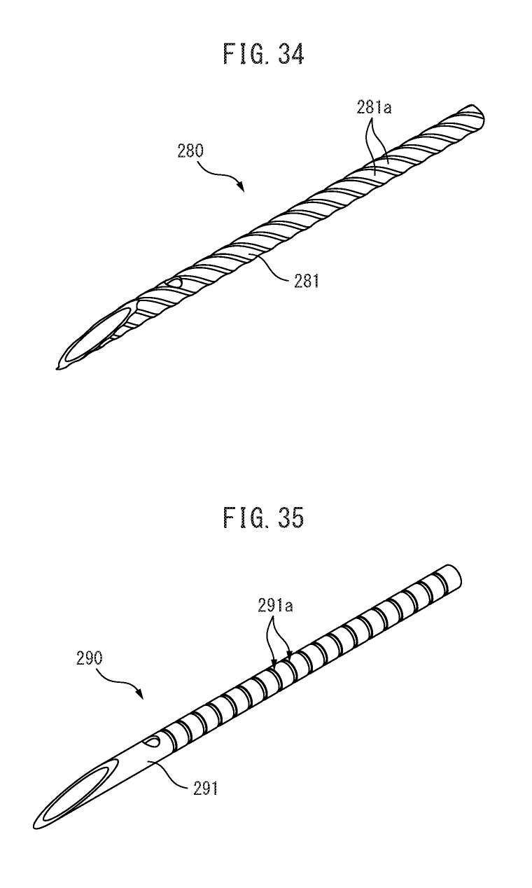

FIG. 34 is a perspective view of a guide needle 281 in a projected state in an electrical surgical instrument 280 according to a 28th embodiment. The surface of the guide needle 281 is formed with a spiral groove 281a.

FIG. 35 is a perspective view or a guide needle 291 in a projected state in an electrical surgical instrument 290 according to a 29th embodiment. The surface of the guide needle 291 is formed with a plurality of ring-shaped grooves 291a.

By the guide needle having a plurality of dimples 261a, a plurality of projections 271a, a spiral groove 281a, a plurality of ring-shaped grooves 291a, or the like, when using the ultrasonic image diagnosis device 6 to observe the position and posture of the guide needle 311 inside the body, the guide needle 311 shown more clearly on the display of the ultrasonic image diagnosis device 6.

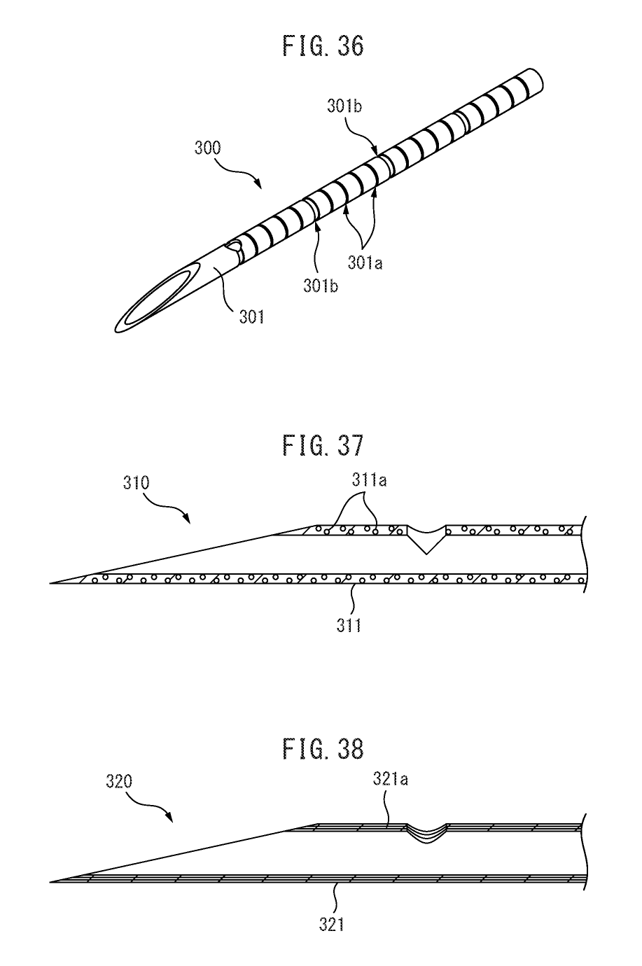

FIG. 36 is a perspective view of a guide needle 301 in a projected state in an electrical surgical instrument 300 according to a 30th embodiment. At the surface of the guide needle 301, a plurality of ring-shaped grooves 301a are formed. These plurality of ring-shaped grooves 301a are formed at equal intervals. These intervals are for example 1 mm. In this case, it is also possible to make the deeper ring-shaped grooves 301b at 5 mm increments. By forming the grooves at equal intervals, it is possible to utilize them as graduations for measuring the depth of insertion of the guide needle 301 into the body.

FIG. 37 is a vertical cross-sectional enlarged view of a guide needle 311 in a projected state in an electrical surgical instrument 310 according to a 31st embodiment. In the wall forming the guide needle 311, a plurality of fine cavities 311a are formed. Due to the plurality of fine cavities 311a in the wall of the guide needle 311, when using the ultrasonic image diagnosis device 6 to observe the position and posture of the guide needle 311 inside the body, the guide needle 311 shown more clearly on the display of the ultrasonic image diagnosis device 6.

FIG. 38 is a vertical cross-sectional enlarged view of a guide needle 321 in a projected state in an electrical surgical instrument 320 according to a 32nd embodiment. The guide needle 321 is formed by a multilayer structure wall 321a. Due to the multilayer structure wall 321a of the guide needle 311, when using the ultrasonic image diagnosis device 6 to observe the position and posture of the guide needle 311 inside the body, the guide needle 311 is shown more clearly on the display of the ultrasonic image diagnosis device 6.

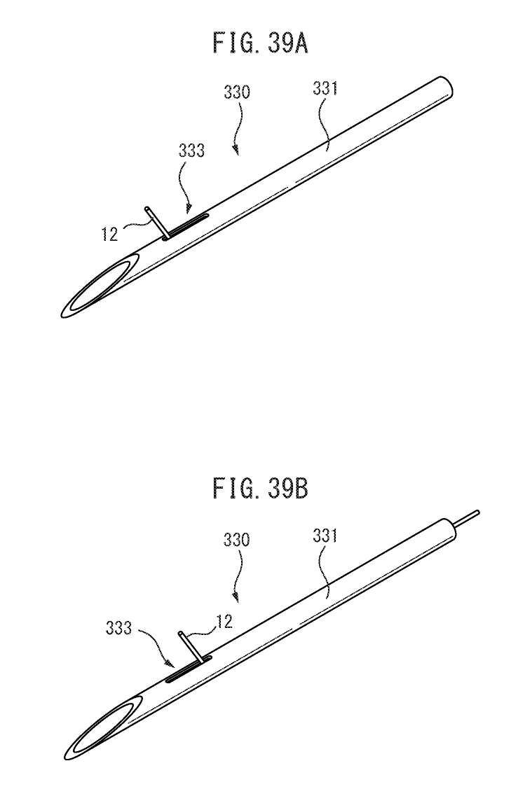

FIG. 39A is a perspective view of a guide needle 331 in a first projected state in an electrical surgical instrument 330 according to a 33rd embodiment, while FIG. 39B is a perspective view of the guide needle 331 in a second projected state in the electrical surgical instrument 330 according to the 33rd embodiment. In the present embodiment, at the side surface of the guide needle 331, an elongated rail open part 333 extending in the longitudinal direction is formed. By the guide needle 331 having the rail open part 333, it is possible to pull the electrode 12 without pulling the guide needle 331 so as to cut the target tissue T. The electrode 12 is guided by the rail open part 333, so the electrode can cut more accurately.

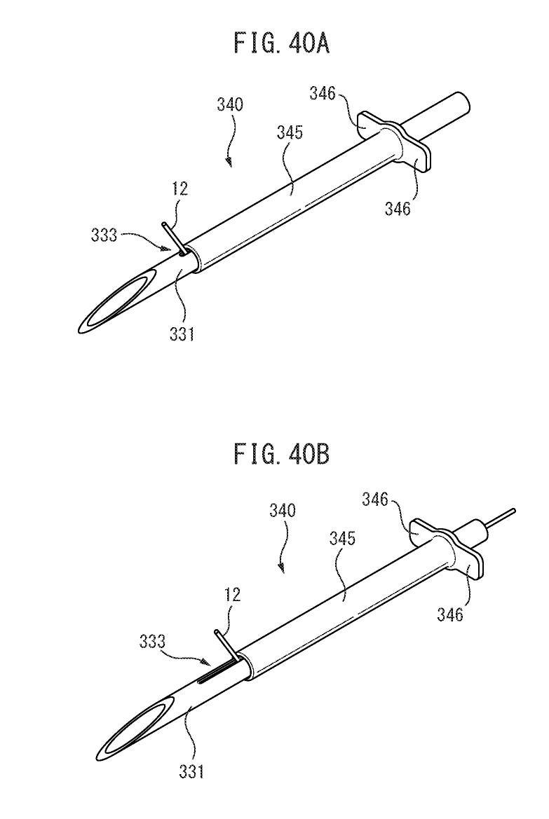

FIG. 40A is a perspective view of a guide needle 331 in a projected state in an electrical surgical instrument 340 according to a 34th embodiment, while FIG. 40B is another perspective view of the guide needle 331 in the projected state in the electrical surgical instrument 340 according to the 34th embodiment. In the present embodiment, around the guide needle 331 of the 33rd embodiment, a tubular member comprised of a sleeve member 345 is attached. The sleeve member 345 has an operating part 346 extending in the radial direction. The sleeve member 345 can be made to slide in the axial direction with respect to the guide needle 331 by gripping the operating part 346. The sleeve member 345 can cover part of the rail open part 333 to adjust the length in the longitudinal direction of the rail open part 333 which is opened in accordance with the size of the target tissue T. The movement of the operating part and the electrode may be mechanically connected.

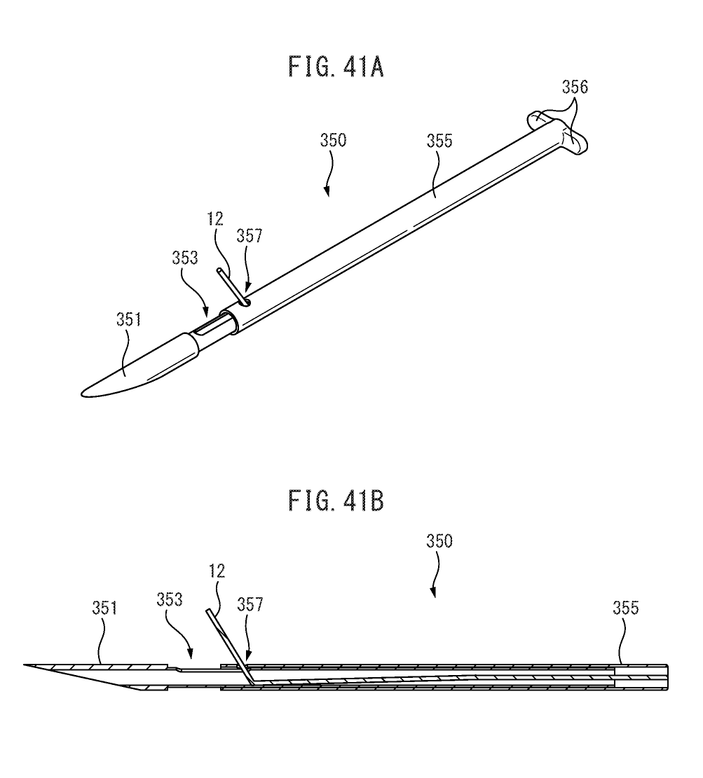

FIG. 41A is a perspective view of a guide needle 351 in a projected state in an electrical surgical instrument 350 according to a 35th embodiment, while FIG. 41B is a vertical cross-sectional view of a guide needle 351 in a projected state in the electrical surgical instrument 350 according to the 35th embodiment. In the present embodiment, around the guide needle 351, a tubular member comprised of a sleeve member 355 is attached. The sleeve member 355 has an operating part 356 extending in the radial direction. The sleeve member 355 can be made to slide in the axial direction with respect to the guide needle 351 by gripping the operating part 356. At the side surface of the guide needle 351, an elongated open part 353 extending in the longitudinal direction is formed. At the side surface of the sleeve member 355, an approximately circular open part 357 is formed. Therefore, by making the sleeve member 355 slide, it is possible to arrange the open part 357 through which the electrode 12 projects out at any position of the elongated open part 353 of the guide needle 351.

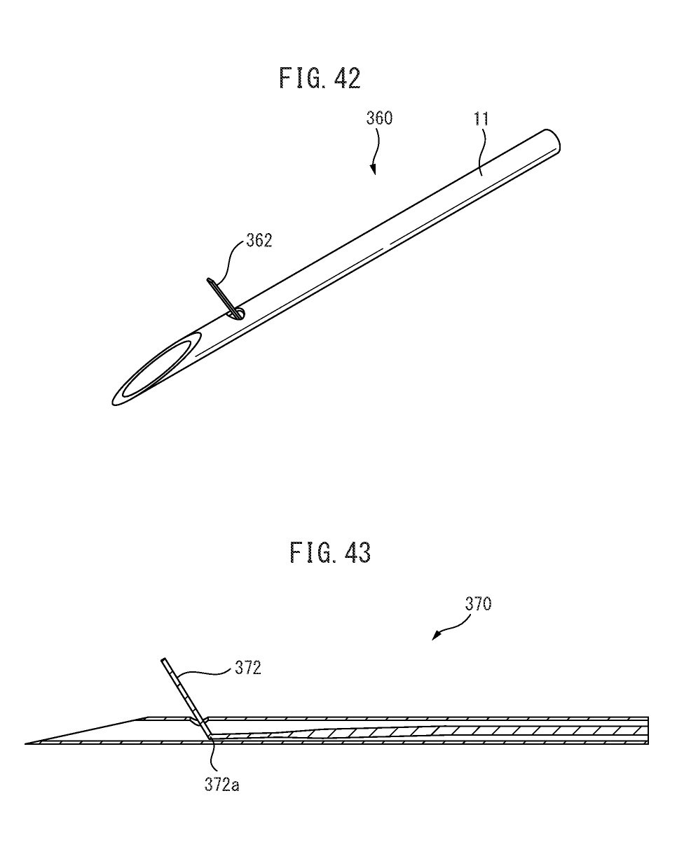

FIG. 42 is a perspective view of a guide needle 11 in a projected state in an electrical surgical instrument 360 according to a 36th embodiment. In the present embodiment, the electrode 362 is formed as a thin strip. Therefore, in the projected state, rotation of the electrode 362 about the axis of the guide needle 11 is reduced due to increased stiffness that resists torsion.

FIG. 43 is a vertical cross-sectional view of a guide needle 11 in a projected state in an electrical surgical instrument 370 according to a 37th embodiment. In the present embodiment, the electrode 372 has a bent part 372a. At least part of the electrode 372 at the proximal side from the bent part 372a is formed thicker than the other portions. Due to this, the rigidity in the axial direction rises and it is possible to prevent unintentional bending or curving of the electrode 372 inside the guide needle 11.

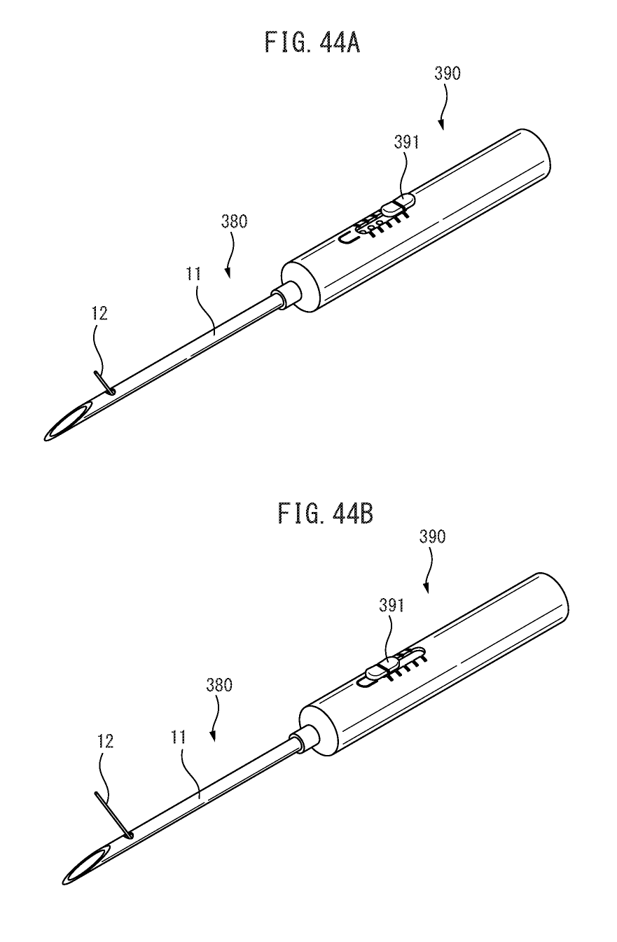

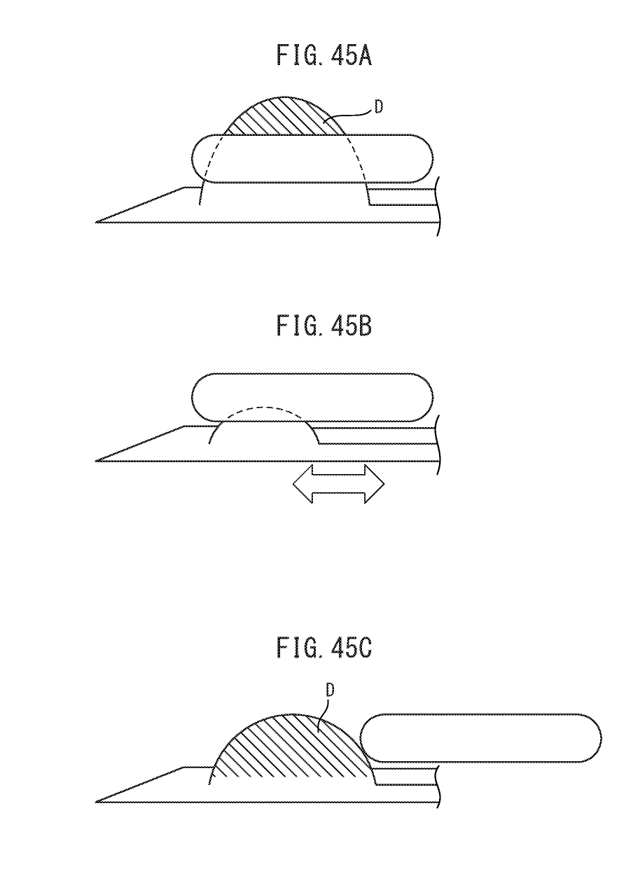

FIG. 44A is a perspective view of a guide needle 11 in a projected state in an electrical surgical instrument 380 according to a 38th embodiment, while FIG. 44B is another perspective view of the guide needle 11 in a projected state in the electrical surgical instrument 380 according to the 38th embodiment. In the present embodiment, the electrical surgical instrument 380 may further have a projection adjustment mechanism 390. The projection adjustment mechanism 390 has an adjustment slider 391 connected to the electrode 12 at the inside. By making the adjustment slider 391 slide in the axial direction, it is possible to adjust the amount of projection of the electrode 12 in the projected state in increments or continuously and further possible to fix the amount of projection.

In the above-mentioned embodiments, there was a single electrode, but there may also be two or more. In those cases, there may be two or more open parts corresponding to the guide needles. Further, as for the material of the electrode, a shape memory alloy may be used. By using a shape memory alloy, it is also possible to use the heat generated by conduction of current to the electrode to deliberately make the electrode deform. Further, it is also possible to use an electrode to measure the impedance etc. of the tissue. Further, it is also possible to provide electrical stimulus through the electrode or guide needle to confirm the presence of nearby nerve tissue or muscle tissue. It is also possible to use the guide needle to suck up body fluids or inject medicine.

The electrical surgical system 1 may further have a device detecting and displaying the angle of insertion of the guide needle into the body, a device able to measure the temperature so as to evaluate the damage to the tissue surrounding the target tissue, a device having a hardness sensor for identifying surrounding tissue, a device having a pressure or force sensor for judging if treatment has been suitably completed, or a device for detecting or displaying the amount of deformation of the guide needle for preventing breakage of the guide needle.

In this Description, various embodiments were explained, but the present invention is not limited to the various embodiments explained above. Please recognize that various changes can be made within the scope described in the following claims.

What is claimed is:

1. An electrical surgical system for cutting or cauterizing tissue,
said electrical surgical system comprising
a tubular member having at least one open part and a front end part able to be inserted through the skin and
at least one electrode able to cut or cauterize tissue configured to be able to project to the outside of said tubular member and to be able to be retracted to the inside of the tubular member through the at least one open part,
in the state where the at least one electrode projects out, a distal end of the at least one electrode being arranged at a position separated from the longitudinal axis of the tubular member.

2. The electrical surgical system according to claim 1, wherein in the state where the at least one electrode projects out, the at least one electrode extends in a radial direction, distal direction, or proximal direction.

3. The electrical surgical system according to claim 1, wherein the at least one open part is formed at a side surface of the tubular member.

4. The electrical surgical system according to claim 1, wherein an amount of the projection of the at least one electrode can be adjusted.

5. The electrical surgical system according to claim 1, wherein at the inside of the tubular member, a guide part is formed configured to guide the at least one electrode in a direction toward the at least one open part at the time of projection of the at least one electrode.

6. The electrical surgical system according to claim 1, wherein the tubular member has a limiting part preventing rotation of the at least one electrode generally about an axis of the tubular member in the state where the at least one electrode projects out.

7. The electrical surgical system according to claim 1, wherein at least part of the at least one electrode projecting out to the outside of the tubular member is insulated.

8. The electrical surgical system according to claim 1, wherein graduations are formed at the outside surface of the tubular member in a generally longitudinal direction.

9. The electrical surgical system according to claim 1, wherein a plurality of surface features, recessed parts, and/or projecting parts are formed as part of the tubular member.

10. The electrical surgical system according to claim 1, wherein the at least one open part is formed so as to enable movement of the at least one electrode along a generally longitudinal direction of the tubular member in the state where the at least one electrode projects out.

11. The electrical surgical system according to claim 1, further comprising an ultrasonic image diagnosis device.

12. The electrical surgical system according to claim 1, wherein the projected out position of the at least one electrode is movable.

13. The electrical surgical system according to claim 1, wherein the range of the gauge of the tubular member is 16G to 25G, preferably 18G to 23G.

14. The electrical surgical system according to claim 1, wherein the length of the tubular member extends from the handle 20 mm to 150 mm.

15. The electrical surgical system according to claim 1, wherein the at least one electrode consists of one or more of stainless steel, tungsten., and shape memory alloy metal.

16. The electrical surgical system according to claim 1, wherein the at least one electrode is a wire-shaped member.

17. The electrical surgical system according to claim 1, wherein the shape of the at least one electrode is formed as a thin strip.

18. The electrical surgical system according to claim 1, wherein the at least one electrode has at least one curved part.

19. The electrical surgical system according to claim 1, wherein the at least one electrode has at least one bent part.

20. The electrical surgical system according to claim 1, wherein the at least one electrode has a bent part near the distal end.

21. The electrical surgical system according to claim 1, wherein the at least one electrode extends up to 20 mm radially from the outside of the tubular member.

22. The electrical surgical system according to claim 1, wherein the range of the diameter of the at least one electrode is 0.1 mm to 0.5 mm.

23. A method for cutting and/or cauterizing tissue, the method comprising
making a tubular member pierce a predetermined location,
arranging the tubular member near the target tissue,
making a distal end of at least one electrode able to cut or able to cauterize tissue and configured to be able to project to the outside of the tubular member and to be able to be retracted to the inside of the tubular member through at least one open part of the tubular member, where the projected part is arranged at a position separated from the longitudinal axis of the tubular member, and
running high frequency current through the at least one electrode.

24. The method for cutting or cauterizing tissue according to claim 23, wherein said projected out orientation is controllable.

25. The method for cutting or cauterizing tissue according to claim 23, further comprising using an ultrasonic image diagnosis device.

26. The method for cutting or cauterizing tissue according to claim 23, wherein the target tissue is ligament and/or fibrous tissue.

27. The method for cutting or cauterizing tissue according to claim 23, wherein the tubular member contains features to identify the location and/or the position using an imaging and/or a sensing device such as ultrasonic, visual, optical, and/or auditory.
